(19) United States
(12) Patent Application Publication
(10) Pub. No.: US 2015/0005600 A1
(43) Pub. Date: Jan. 1, 2015

(54) FINGER-PLACEMENT SENSOR TAPE

(71) Applicant: Cercacor Laboratories, Inc., Irvine, CA (US)

(72) Inventors: Thomas B. Blank, Laguna Beach, CA (US); Hung The Vo, Fountain Valley, CA (US); Cristiano Dalvi, Lake Forest, CA (US); Sean Merritt, Lake Forest, CA (US); Marcelo M. Lamego, Cupertino, CA (US)

(21) Appl. No.: 14/210,410

(22) Filed: Mar. 13, 2014

Related U.S. Application Data
(60) Provisional application No. 61/778,446, filed on Mar. 13, 2013.

(51) Int. Cl. A61B 5/00; A61B 5/145; A61B 5/1455

(52) U.S. Cl. CPC A61B 5/6833; A61B 5/14552; A61B 5/14532
USPC 600/344

(57) ABSTRACT

A finger-placement sensor tape aligns and removably secures a finger to a sensor pad of a reusable finger-clip optical sensor so as to assure the finger is repeatably aligned between the sensors emitters and detectors and that the finger stays aligned during a test procedure. The sensor tape has a double-sided tape layer having a silicon-based adhesive on a finger side and an acrylic adhesive on a sensor-side. An aperture is defined in the tape layer so as to allow emitters disposed in a top sensor pad to emit light through the tape layer to detectors disposed in a bottom sensor pad. A finger-side release layer is removably disposed over the silicon-based adhesive. A sensor-side release layer is removably disposed over the acrylic adhesive.

### PRIORITY CLAIM TO RELATED PROVISIONAL APPLICATION

The present application claims priority benefit under 35 U.S.C. §119(e) to U.S. Provisional Patent Application Ser. No. 61/778,446 filed Mar. 13, 2013, titled Finger-Placement Sensor Tape. The above-cited provisional patent application is hereby incorporated in its entirety by reference herein.

### BACKGROUND OF THE INVENTION

Noninvasive physiological monitoring systems for measuring constituents of circulating blood have advanced from basic pulse oximeters capable of measuring blood oxygen saturation to advanced blood parameter monitors capable of measuring various blood constituents. A basic pulse oximeter typically includes an optical sensor, a monitor for processing sensor signals and displaying results and a cable electrically interconnecting the sensor and the monitor. A basic pulse oximetry sensor typically has a red wavelength light emitting diode (LED), an infrared (IR) wavelength LED and a photodiode detector. The LEDs and detector are attached to a patient tissue site, such as a finger. The cable transmits drive signals from the monitor to the LEDs, and the LEDs respond to the drive signals to transmit light into the tissue site. The detector generates a photoplethysmograph signal responsive to the emitted light after attenuation by pulsatile blood flow within the tissue site. The cable transmits the detector signal to the monitor, which processes the signal to provide a numerical readout of oxygen saturation (SpO₂) and pulse rate, along with an audible pulse indication of the person's pulse. The photoplethysmograph waveform may also be displayed.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, Calif. and are incorporated in their entirety by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entirety by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are capable of measuring various blood parameters in addition to SpO₂, such as total hemoglobin and carboxyhemoglobin to name a few. Advanced blood parameters measurement systems are described in at least U.S. Pat. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. No. 7,957,780, filed Mar. 1, 2006, titled Physiological Parameter Confidence Measure and U.S. Pat. No. 8,224,411, filed Mar. 1, 2006, titled Noninvasive Multi-Parameter Patient Monitor, all assigned to Cercacor Laboratories, Inc., Irvine, Calif. (“Cercacor”) and all incorporated in their entirety by reference herein. An advanced parameter measurement system that includes acoustic monitoring is described in U.S. Pat. Pub. No. 2010/0274099, filed Dec. 21, 2009, titled Acoustic Sensor Assembly, assigned to Masimo and incorporated in its entirety by reference herein.

Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad87™ and Rad57™ monitors, all available from Masimo. Advanced parameter measurement systems may also include acoustic monitoring such as acoustic respiration rate (RRa™) using a Rainbow Acoustic Sensor™ and Rad87™ monitor, available from Masimo. Such advanced pulse oximeters, low noise sensors and advanced parameter systems have gained rapid acceptance in a wide variety of medical applications, including surgical wards, intensive care and neonatal units, general wards, home care, physical training, and virtually all types of monitoring scenarios.

### SUMMARY OF THE INVENTION

A finger-placement sensor tape aligns and removably secures a finger to a sensor pad of a reusable finger-clip optical sensor so as to assure the finger is repeatably aligned between the sensors emitters and detectors and that the finger stays aligned during a test procedure. The sensor tape has a double-sided tape layer having a silicon-based adhesive on a finger side and a abrasive adhesive on a sensor-side. An aperture is defined in the tape layer so as to allow emitters disposed in a top sensor pad to emit light through the tape layer to detectors disposed in a bottom sensor pad. A finger-side release layer is removably disposed over the silicon-based adhesive. A sensor-side release layer is removably disposed over the abrasive adhesive. The finger-placement sensor tape is installed on one or both sensor pads of the finger-clip sensor. The sensor-side release layer is firstly removed from the tape layer so as to dispose the tape layer within a finger bed portion of a sensor pad. The finger-side release layer is secondly removed from the tape layer so as to adhere a finger to the tape layer and within the finger bed accordingly.

In various embodiments, the finger-placement sensor tape has a peel tab extending from the sensor-side release layer so as to facilitate removal of the sensor-side release layer from the tape layer. The double-sided tape layer adhesively attaches to a sensor pad via the second adhesive after removal of the sensor-side release layer. A finger attaches to the double-sided tape layer via the first adhesive after removal of the finger-side release layer. The first adhesive is a silicon-based adhesive and the second adhesive is an acrylic-based adhesive. A roll liner is removably disposed on an adhesive-side of the finger-side release layer. An applicator attaches to the adhesive-side of the finger-side release layer after removal of the roll liner so as to facilitate placement of the double-sided tape layer within a reusable finger-clip sensor. A pull tab extends from the finger-side release layer so as to facilitate removal of the finger-side release layer after placement of the double-sided tape layer within a reusable finger-clip sensor.

An aspect of a finger-placement sensor is a method for securely adhering a fingertip within a reusable optical sensor having a top sensor pad and a bottom sensor pad urged proximate the fingertip in a closed sensor position. A finger-placement sensor method comprises configuring a double-sided tape layer having a first adhesive on a finger side and a second adhesive on a sensor-side, disposing an aperture in the tape layer so as to allow emitters disposed in a top sensor pad to emit light through the tape layer to detectors disposed in a bottom sensor pad, removing a sensor-side release layer disposed over the second adhesive and adhesively attaching the tape layer within a finger bed portion of the bottom sensor pad.

In various embodiments, the finger-side release layer is removed from the tape layer so as to so as to expose the first adhesive, enabling the first adhesive to adhere a fingertip to the tape layer within the finger bed portion accordingly. A third adhesive is disposed on the finger-side release layer. A liner is removably disposed over the third adhesive. The liner is removed to expose the third adhesive. An applicator is attached to the third adhesive. The applicator is used to attach the double-sided tape layer within the finger bed. The applicator and attached finger-side release layer are rolled off of the double-sided tape layer and a finger tip is removably adhered to the exposed first adhesive.

Another aspect of a finger-placement sensor tape comprises a double-sided tape means for removably adhering a fingertip within a finger bed portion of a reusable, clip-on optical sensor. A first adhesive means adheres the tape means to the finger bed disposed on a sensor-side of the tape means. A first release layer means is disposed on the sensor-side of the tape means over the first adhesive means. The first release layer means is removed from the tape means so as to expose the first adhesive means and so as to attach the double-sided tape means to the finger bed portion of the optical sensor.

In various embodiment, a second adhesive means is disposed on a finger-side of the tape means. A second release layer means is disposed on the finger-side of the tape means over the second adhesive means and the second release layer removed from the tape means so as to expose the second adhesive means and secure a fingertip to the tape means and within the finger bed portion of the optical sensor. A third adhesive is disposed on a finger-side of the second release layer means and a third liner means disposed over the third adhesive. An applicator means for attaching the double-sided tape means to the finger bed portion is adhered to the third adhesive after removal of the third liner. A pull tab means extends from the second release layer means.

### BRIEF DESCRIPTION OF THE DRAWINGS

FIG. 1 is a perspective illustration of a double-sided adhesive sensor tape applied to a sensor bottom pad so as to establish and maintain repeatable finger placement within a reusable optical sensor;

FIGS. 2A-C are top, side and exploded side views, respectively, of a double-sided adhesive sensor tape embodiment;

FIGS. 3A-C are exploded side, exploded perspective and perspective views, respectively, of a double-sided adhesive sensor tape embodiment and a corresponding tape applicator for applying the sensor tape to a sensor pad;

FIGS. 4A-B are top and exploded perspective views, respectively, of a double-sided adhesive sensor tape embodiment having an integrated pull tab;

FIG. 5 is a perspective view of a sensor tape integrated pull tab that allows easy removal of a finger-side release layer after the sensor tape is attached to a sensor pad and just prior to finger placement and adhesive attachment to the sensor pad; and

FIGS. 6A-B are perspective views of a sensor tape applicator threaded with the sensor tape integrated pull tab positioned on a sensor bottom pad and the applied sensor tape after removal of the finger-side release layer to expose the finger placement adhesive.

### DETAILED DESCRIPTION OF THE PREFERRED EMBODIMENTS

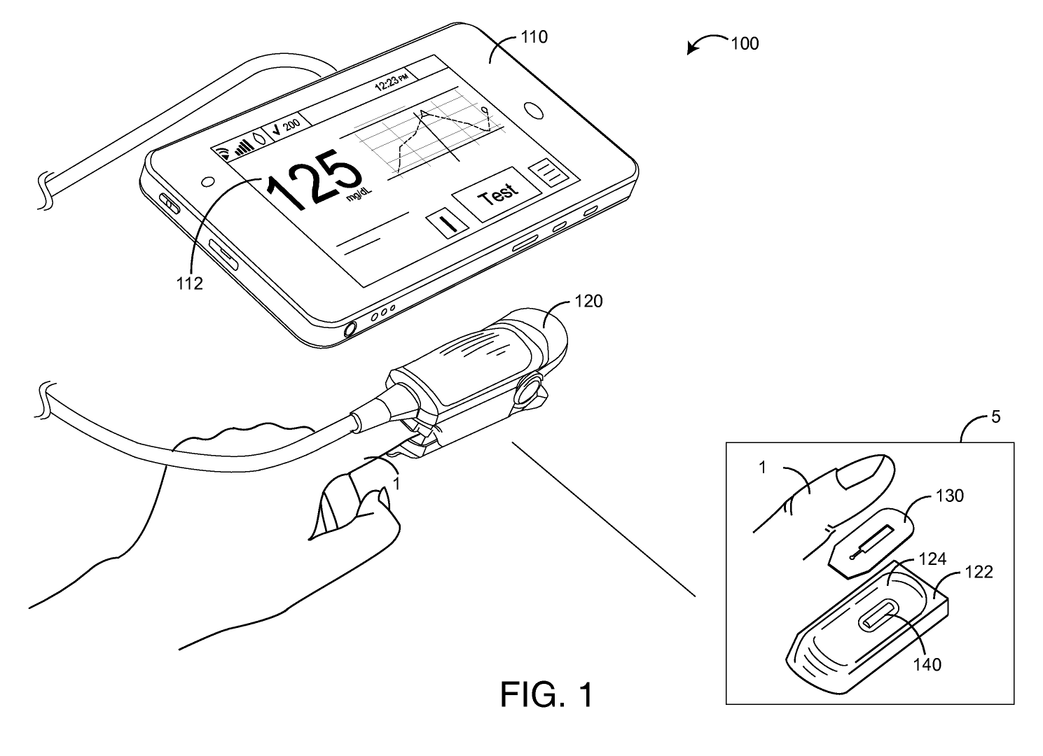

FIG. 1 generally illustrates a physiological monitoring system 100 that utilizes a finger-placement sensor tape 130. The monitoring system 100 includes a blood parameter monitor 110 and an optical sensor 120 configured to noninvasively measure and display a patient's blood glucose level among other parameters. In an embodiment, the sensor 120 attaches to a person's finger 1 so as to illuminate the finger with optical radiation, which is detected after attenuation by fingertip blood flow. The sensor communicates these optical measurements of blood attenuation, along with other sensor data such as sensor position and temperature, to the monitor 110. The monitor 110 calculates and displays blood parameter measurements 112 accordingly. A physiological monitoring system is described in U.S. patent application Ser. No. 13/308,461 titled Handheld Processing Device Including Medical Applications For Minimally And Non Invasive Glucose Measurements, filed Nov. 30, 2011, assigned to Cercacor Laboratories Inc. (“Cercacor”), and incorporated in its entirety by reference herein. A reusable optical sensor is described in U.S. patent application Ser. No. 13/473,477 titled Personal Health Device, filed May 16, 2012, assigned to Cercacor and incorporated in its entirety by reference herein.

As shown in FIG. 1, and in particular inset 5 therein, optical sensor measurements as described above are sensitive to finger placement between the top and bottom sensor pads. A finger-placement sensor tape 130 is a double-sided adhesive tape applied to one or both of the top sensor pad and bottom sensor pad 122 so as to advantageously improve finger placement stability and repeatability with respect to the finger pad(s) 122 and improve optical sensor measurements accordingly.

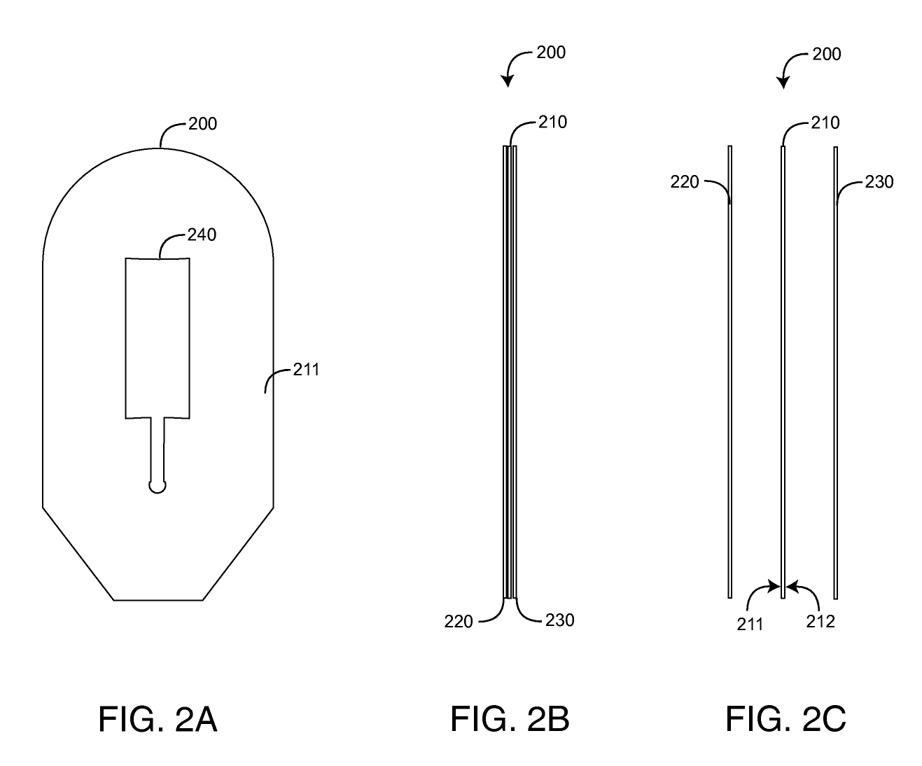

FIGS. 2A-C illustrate a finger-placement sensor tape embodiment 200 (“sensor tape”) having a double-sided adhesive tape layer 210 (“tape layer”), a finger-side release layer 220 and a pad-side release layer 230. The tape layer 210 has a silicon-based glue on a finger-side 211 and an acrylic or abrasive glue on a pad-side 212. In an embodiment, the tape layer 210 is a double-coated silicon adhesive tape such as 3M 2477 or equivalent. An optical path aperture 240 is disposed through the sensor tape 200 so as to expose a detector lens located proximate a bottom sensor pad 122. As such, LED light emitted from a top sensor pad (not shown) enters the fingernail bed and exits the fingertip so as to reach sensor detectors via the aperture 240 after attenuation by blood perfused tissue and pulsatile blood flow within the fingertip.

Also shown in FIGS. 1-2, the tape layer 210 is applied to the sensor pad 122 by removing the pad-side release layer 230, aligning the tape layer 210 to the sensor finger bed 124 and adhering the pad side 212 within the finger bed 124. The finger 1 is then aligned within the finger bed 124 by removing the finger-side release layer 220, positioning the finger over the finger bed 124 and pressing the finger against the tape layer finger-side 211 so as to removably adhere the finger within the finger bed 124. This procedure advantageously allows a person to visually align and adhere their finger properly within the finger bed 124 while the sensor clip 120 is open. This procedure also provides assurance that a finger 1 stays properly aligned to the sensor pad 122 after the sensor clip 120 is closed, during movement of a hand to a comfortable position for testing, and during a test procedure itself.

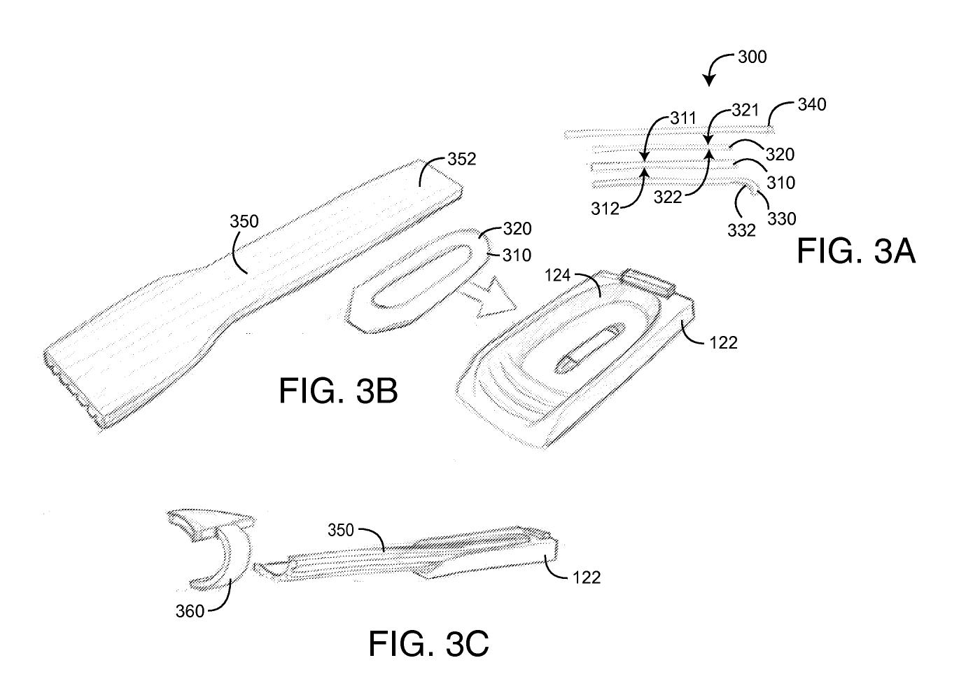

FIG. 3A illustrates a finger-placement sensor tape 300 (“sensor tape”) embodiment having a double-sided adhesive tape layer 310 (“tape layer”), a finger-side release layer 320, a sensor-side release layer 330 and a roll liner 340. As above, the tape layer 310 has a silicon-based glue on a finger-side 311 and an acrylic or abrasive glue on a pad-side 312. Advantageously, the finger-side release layer 320 has an adhesive side 321 for adherence to an applicator 350 (FIG. 3B) and a release side 322. In an embodiment, the sensor-side release layer 330 has a finger tab 332 as an aid to separate the release layer 330 from the tape layer 310.

FIG. 3B illustrates a flex fold applicator 350 for applying the sensor tape 310 to a sensor pad 122. Initially, the roll liner 340 is removed from the adhesive side 321 of the finger-side release layer 320 so as to expose the adhesive side 321. Then, the applicator 350 is adhered to the exposed adhesive side 321 so as to attach the remainder of the sensor tape 300 to the applicator 350. Next, the finger tab 332 is grasped and the sensor-side release layer 330 is removed. The applicator 350 is then used to position the tape layer 310 and release layer 320 within the sensor clip 120 (FIG. 1) and over the sensor pad 122. The applicator 350 then applies this combination 310, 320 to the finger bed 124.

In an embodiment, the applicator 350 length and width are sized so as to center the sensor tape 310 within the sensor pad cavity 124. In an embodiment, the applicator tip 352 is also shaped to precisely fit the sensor pad cavity 124. The applicator 350 advantageously acts as a precision guide for sensor tape 300 attachment to the sensor pad 122, which provides further consistency as to subsequent finger placement within the sensor pad 122. Applicator 350 use is particularly advantageous for applying a sensor tape 310 to a large sensor pad 122.

As shown in FIG. 3C, once the sensor tape 310 and applicator 350 are firmly positioned in the sensor pad cavity 124, pressure is applied at the applicator tip 352 to insure the tape layer pad-side 312 adheres to the sensor pad 122. Finally, the flex fold applicator 350 is manually curled 360 so as to peel the release layer 320 from the tape layer 310, leaving just the tape layer 310 and its exposed finger-side 311 adhesive within the sensor pad 122, ready for finger placement.

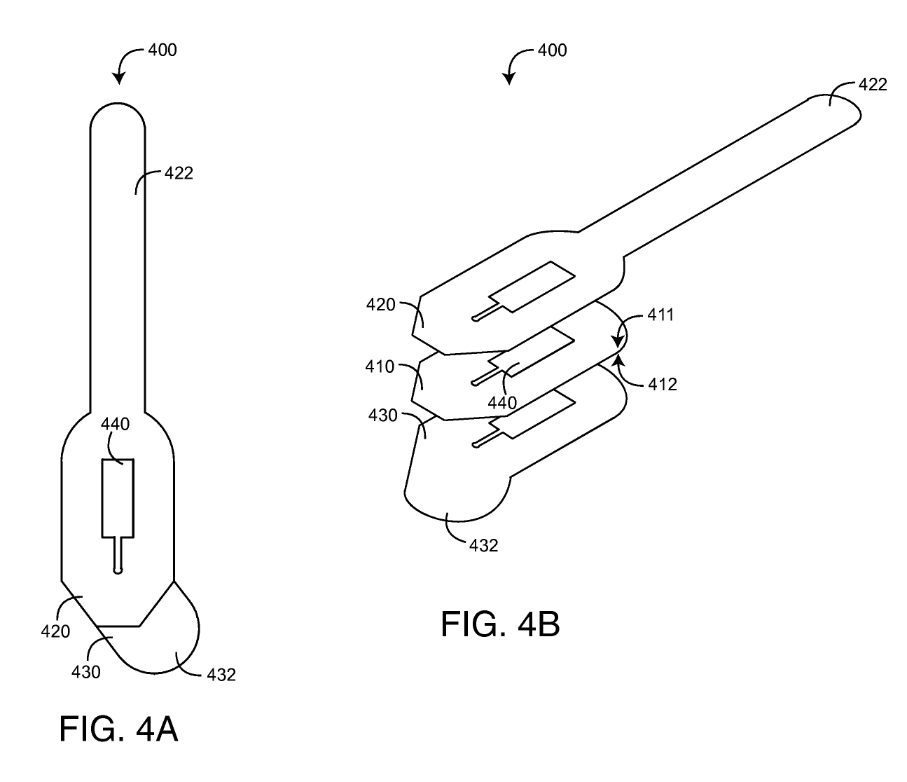

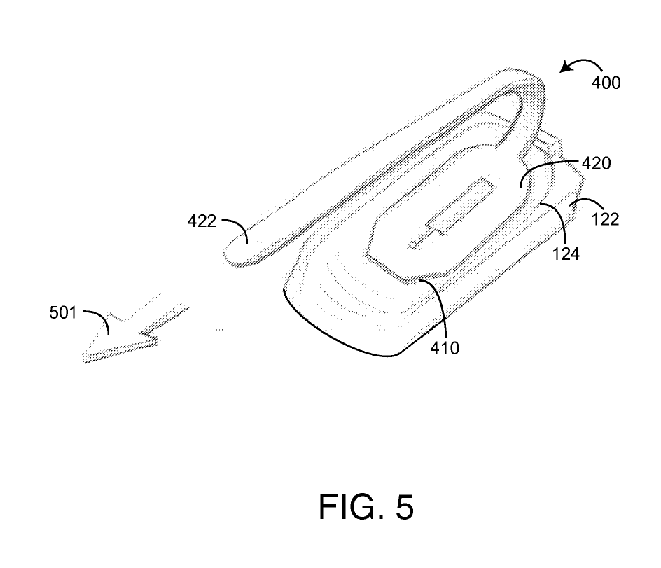

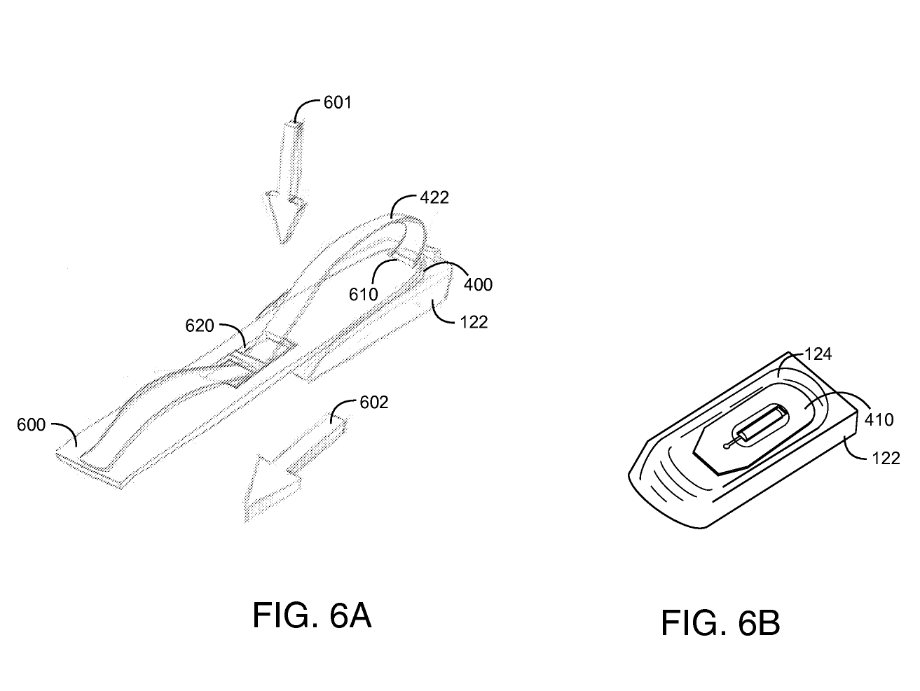

FIGS. 4A-B illustrate a finger-placement sensor tape embodiment 400 (“sensor tape”) having an integrated pull tab 422. The sensor tape 400 has a double-sided adhesive tape layer 410 (“tape layer”), a finger-side release layer 420 and a pad-side release layer 430. Advantageously, the pull tab 422 extends outside of the sensor 120 (FIG. 1) at a finger entry point, eliminating the need to reach inside the sensor so as to peel off the finger-side release layer and potentially encounter the finger-side adhesive on the release layer in the process. A peel tab 432 extends from the pad-side release layer 430 allowing that layer to be gripped for easy removal from a pad-side 412 of the tape layer 410. The tape layer 410 has a silicon-based glue on a finger-side 411 and an acrylic or abrasive glue on the pad-side 412. An optical path aperture 440 is disposed at least through the tape layer 410 so as to allow light to illuminate a finger 1, as described with respect to FIG. 1, above. Sensor tape 400 installation is described with respect to FIGS. 5-6, below.

FIG. 5 illustrates the use of a sensor tape pull tab 422, as described with respect to FIGS. 4A-B, above, to removably secure a finger to a sensor pad 122. The tape layer 410 is applied to the sensor pad 122 by removing the pad-side release layer 430 (FIGS. 4A-B), aligning the tape layer 410 to the sensor finger bed 124 and adhering the pad side 412 (FIG. 4B) within the finger bed 124. Once the tape layer 410 is adhered to a sensor pad 122, the pull tab 422 is grasped and pulled out 501 the open entrance of the finger clip sensor 120 (FIG. 1), exposing the tape layer 410 adhesive and readying the sensor 120 (FIG. 1) for finger placement.

FIGS. 6A-B illustrate a tape applicator 600 advantageously threaded with an integrated pull tab 422 (FIGS. 4A-B) so as to conveniently apply a sensor tape 400 to a sensor pad 122 and remove the sensor pad release layer 420 (FIG. 5) via an open sensor clip 120 (FIG. 1). As shown in FIG. 6A, the pull tab 422 is weaved through applicator slots 610, 620. A pad-side release layer 430 (FIGS. 4A-B) is removed, and the applicator 600 is pressed 601 into the sensor pad 122 so as to adhere the tape layer 410 within the sensor cavity 124. Then, while pushing down 601, the applicator 600 is pulled out and off 602 of the sensor pad 122. As shown in FIG. 6B, this leaves the tape layer 410 adhered to the sensor pad 122 and ready for finger placement.

A finger-placement sensor tape has been disclosed in detail in connection with various embodiments. These embodiments are disclosed by way of examples only and are not to limit the scope of the claims that follow. One of ordinary skill in art will appreciate many variations and modifications.

What is claimed is:

1. A finger-placement sensor tape aligns and removably secures a finger to a sensor pad of a reusable finger-clip optical sensor so as to assure the finger is repeatably aligned between the sensor emitters and detectors and that the finger stays aligned during a test procedure, the finger-placement sensor tape comprising:
a double-sided tape layer having a first adhesive on a finger side and a second adhesive on a sensor-side;
an aperture defined by the tape layer so as to allow emitters disposed in a top sensor pad to emit light through the tape layer to detectors disposed in a bottom sensor pad;
a finger-side release layer removably disposed over the first adhesive;
a sensor-side release layer removably disposed over the second adhesive;
the sensor-side release layer firstly removed from the tape layer so as to adhesively attach the tape layer within a finger bed portion of a sensor pad; and
the finger-side release layer secondly removed from the tape layer so as to adhere a finger to the tape layer and within the finger bed accordingly.

2. The finger-placement sensor tape according to claim 1 further comprising a peel tab extending from the sensor-side release layer so as to facilitate removal of the sensor-side release layer from the tape layer.

3. The finger-placement sensor tape according to claim 2 wherein the double-sided tape layer adhesively attaches to a sensor pad via the second adhesive after removal of the sensor-side release layer.

4. The finger-placement sensor tape according to claim 3 wherein a finger attaches to the double-sided tape layer via the first adhesive after removal of the finger-side release layer.

5. The finger-placement sensor tape according to claim 4 wherein the first adhesive is a silicon-based adhesive and the second adhesive is an acrylic-based adhesive.

6. The finger-placement sensor tape according to claim 5 further comprising a roll liner removably disposed on an adhesive-side of the finger-side release layer.

7. The finger-placement sensor tape according to claim 6 further comprising an applicator that attaches to the adhesive-side of the finger-side release layer after removal of the roll liner so as to facilitate placement of the double-sided tape layer within a reusable finger-clip sensor.

8. The finger-placement sensor tape according to claim 5 further comprising a pull tab extending from the finger-side release layer so as to facilitate removal of the finger-side release layer after placement of the double-sided tape layer within a reusable finger-clip sensor.

9. A finger-placement sensor method for securely adhering a fingertip within a reusable optical sensor having a top sensor pad and a bottom sensor pad urged proximate the fingertip in a closed sensor position, the finger-placement sensor method comprising:
configuring a double-sided tape layer having a first adhesive on a finger side and a second adhesive on a sensor-side;
disposing an aperture in the tape layer so as to allow emitters disposed in a top sensor pad to emit light through the tape layer to detectors disposed in a bottom sensor pad;
removing a sensor-side release layer disposed over the second adhesive; and
adhesively attaching the tape layer within a finger bed portion of the bottom sensor pad.

10. The finger-placement sensor method according to claim 9 further comprising removing the finger-side release layer from the tape layer so as to so as to expose the first adhesive, enabling the first adhesive to adhere a fingertip to the tape layer within the finger bed portion accordingly.

11. The finger-placement sensor method according to claim 10 further comprising disposing a third adhesive on the finger-side release layer.

12. The finger-placement sensor method according to claim 11 further comprising removably disposing a liner over the third adhesive.

13. The finger-placement sensor method according to claim 12 further comprising:
removing the liner to expose the third adhesive; and
attaching an applicator to the third adhesive.

14. The finger-placement sensor method according to claim 13 further comprising using the applicator to attach the double-sided tape layer within the finger bed.

15. The finger-placement sensor method according to claim 14 further comprising:
rolling the applicator and attached finger-side release layer off of the double-sided tape layer; and
removably adhering a finger tip on the exposed first adhesive.

16. A finger-placement sensor tape comprising:
a double-sided tape means for removably adhering a fingertip within a finger bed portion of a reusable, clip-on optical sensor;
a first adhesive means for adhering the tape means to the finger bed disposed on a sensor-side of the tape means;
a first release layer means disposed on the sensor-side of the tape means over the first adhesive means;
the first release layer means removed from the tape means so as to expose the first adhesive means and so as to attach the double-sided tape means to the finger bed portion of the optical sensor.

17. The finger-placement sensor tape according to claim 16 further comprising:
a second adhesive means disposed on a finger-side of the tape means;
a second release layer means disposed on the finger-side of the tape means over the second adhesive means; and
the second release layer removed from the tape means so as to expose the second adhesive means and secure a fingertip to the tape means and within the finger bed portion of the optical sensor.

18. The finger-placement sensor tape according to claim 17 further comprising:
a third adhesive disposed on a finger-side of the second release layer means; and
a third liner means disposed over the third adhesive.

19. The finger-placement sensor tape according to claim 18 further comprising an applicator means for attaching the double-sided tape means to the finger bed portion, the applicator means adhered to the third adhesive after removal of the third liner.

20. The finger-placement sensor tape according to claim 19 further comprising a pull tab means extending from the second release layer means.
